(19) United States
(12) Patent Application Publication
(10) Pub. No.: US 2023/0147473 A1
(43) Pub. Date: May 11, 2023

(54) HYPOGLYCEMIA AND/OR HYPERGLYCEMIA DETECTOR

(71) Applicant: LAKKA HEALTH OY, Lempäälä (FI)

(72) Inventors: Sami LAKKA, Lempäälä (FI); Elina VÄLIPAKKA, Tampere (FI); Heidi TOIVANEN, Nokia (FI)

(73) Assignee: LAKKA HEALTH OY, Lempäälä (FI)

(21) Appl. No.: 17/905,097

(22) PCT Filed: Feb. 26, 2021
(86) PCT No.: PCT/FI2021/050145

§ 371 Date: Aug. 26, 2022

(30) Foreign Application Priority Data
Feb. 27, 2020 (FI) 20205199

(51) Int. Cl. A61B 5/145; A61B 5/00

(52) U.S. Cl. CPC A61B 5/145; A61B 5/6833; A61B 5/746

(57) ABSTRACT

Hypoglycemia and/or hyperglycemia detector (100), including an enclosure (110), one or more gas sensors (132) and one or more processors (134). The enclosure (100) includes a bottom surface (112) configured for positioning against skin (10), for example with an adhesive patch (20). The sensors (132) are positioned within the enclosure (110) and configured for detecting gas emissions from the skin (10) for providing one or more measurement signals. The processors (134) are configured for detecting an indication of hypoglycemia and/or an indication of hyperglycemia from the one or more measurement signals for providing an alarm signal when the indication of hypoglycemia and/or the indication hyperglycemia is detected.

### FIELD

The present invention relates to detecting hypoglycemia and/or hyperglycemia.

### BACKGROUND

Hypoglycemia is a state, where blood sugar has fallen below normal. If untreated, this condition can be serious and life threatening for a diabetic person. Consequently, an important factor in diabetes treatment is regular measuring and monitoring of blood sugar levels. Current commercially available glucose monitoring systems are invasive and thus require skin to be penetrated with a needle. Various currently available monitoring systems have offered a great relief to everyday life of a diabetic person but, unfortunately, cannot always be fully utilized due to high price and limited availability. A challenge is also presented by hyperglycemia, which is a condition in which an excessive amount of glucose circulates in the blood plasma.

### OBJECTIVE

An objective is to provide a hypoglycemia and/or hyperglycemia detector for detecting the presence of hypoglycemia and/or hyperglycemia.

In particular, it is an objective to provide a non-invasive and affordable hypoglycemia and/or hyperglycemia detector that can be comfortably used.

Additionally, it is an objective to provide a detector that can provide automatic monitoring and alarming for hypoglycemia and/or hyperglycemia.

Finally, it is an objective to provide a reliable hypoglycemia and/or hyperglycemia detector for all diabetic persons, including both type I and II, regardless of age or living standards.

### SUMMARY

According to a first aspect, a hypoglycemia and/or hyperglycemia detector (herein also “the detector”) comprises an enclosure comprising a bottom surface configured for positioning against skin. This allows the detector to be easily used for continuous monitoring as it can be positioned on the body of a person, for example on an arm of the person (herein, “continuous monitoring” may refer to monitoring with substantially continuous measurements and/or monitoring with repeated measurements, for example once in an hour or more often, the monitoring with repeated measurements, in particular, allowing cost-efficient monitoring). The detector further comprises one or more gas sensors (herein also “the sensors”), such as a volatile organic compound (VOC) sensor, positioned within the enclosure and configured for detecting gas emissions from the skin for providing one or more measurement signals. The positioning of the sensors within the enclosure allows the sensors to be at least partially isolated from ambient gases while being focused on detecting gas emissions from the skin. The detector also comprises one or more processors (herein also “the processors”) configured for detecting an indication of hypoglycemia and/or an indication of hyperglycemia from the one or more measurement signals (herein also “the measurement signals”) for providing an alarm signal when the indication of hypoglycemia and/or the indication of hyperglycemia is detected. This allows detecting hypoglycemia and/or hyperglycemia without necessarily measuring any actual blood sugar levels. Instead, the detection can be performed based on an indication of hypoglycemia and/or an indication hyperglycemia, for example a deviation of the one or more measurement signals from one or more baseline signals.

The detector, or the processors in particular, may be specifically configured for detecting either one of hypoglycemia or hyperglycemia. As an alternative, it may be specifically configured for detecting both hypoglycemia and hyperglycemia. The detector, or the processors in particular, can be specifically configured for detecting changes in the gas emissions, or to determine one or more indications correlating with blood glucose level. This may be done continuously, or repeatedly, to obtain a real-time indication of blood-glucose level. Regardless of whether the actual measurement allows the determination of a continuous indicator for blood-glucose level, the indication of hypoglycemia and/or the indication of hyperglycemia may be provided as discrete categories.

The detector as a whole allows non-invasive detection of hypoglycemia and/or hyperglycemia. Moreover, it allows automatic monitoring of hypoglycemia and/or hyperglycemia, as the processors may be configured for causing the detection to be performed automatically, for example according to a schedule. The detector therefore allows also self-monitoring of an unconscious person, for example during sleep, when blood sugar level may change unexpectedly. This allows particularly efficient monitoring of hypoglycemia and/or hyperglycemia for children. It also allows monitoring without waking up a sleeping person using the detector, unless desired. The detector can be repositioned during continuous use, for example once a day, to reduce stress on the skin. The detector can be configured to provide an alarm signal, when the indication of hypoglycemia and/or the indication of hyperglycemia is detected, which in turn allows an unconscious person using the detector or another person to be alerted for the presence of hypoglycemia and/or hyperglycemia.

One important observation of the present disclosure is that for a hypoglycemia and/or hyperglycemia detector it is enough to detect an indication of hypoglycemia and/or an indication of hyperglycemia. This means that the detector does not need to be able to measure the actual blood sugar levels but only observe changes in measurement signals to indicate the presence of hypoglycemia and/or hyperglycemia. This allows the hypoglycemia and/or hyperglycemia detector to be provided at reduced cost in comparison to more complex devices. For a diabetic person, a blood glucose level below a threshold level 3.9 mmol/1 or 4 mmol/l may be considered indicative of hypoglycemia. In units of mg/dl, a threshold level of 70 mg/dl may also be used, as an upper limit as above. Similarly, a blood glucose level above a threshold level 11 mmol/1 or 11.1 mmol/l may be considered indicative of hyperglycemia. However, even more stringent threshold may be used, for example at 7.0 mmol/1 or 126 mg/dl, as a lower limit as above. This can be used, in particular, for fasting plasma glucose. Instead of utilizing these values, the detector allows hypoglycemia and/or hyperglycemia to be detected as a sudden change in the gas emissions, which are indicative of hypoglycemia and/or hyperglycemia and may develop quickly, even substantially instantly in response to hypoglycemia and/or hyperglycemia. By utilizing the sensors, the change in gas emissions can be translated into a change in the measurement signals. As an example, the change can be quantified utilizing one or more baseline signals that indicate the absence of hypoglycemia and/or hyperglycemia (herein also “the baseline signals”). The detector may also utilize a calibration measurement, when the user is in non-hypoglycemia and/or non-hyperglycemia state, to determine the baseline signals, allowing user-specific calibration of the detector for detecting hypoglycemia and/or hyperglycemia. Comparing any measurement signals to the baseline signals can be used to detect hypoglycemia and/or hyperglycemia, since a deviation from the baseline signals can be accurately interpreted as the indication of hypoglycemia and/or an indication of hyperglycemia. The actual amount of deviation may depend on the particulars of the sensors used, but this can be straightforwardly determined, for example through clinical trials. Similarly, sensitivity of the sensors for gas emissions indicative of hypoglycemia and/or hyperglycemia can be similarly ensured, for example by varying the thickness of sensor materials. Naturally, while the detector allows detection of hypoglycemia and/or hyperglycemia without measurement of actual blood sugar levels, it may also be compounded or used in conjunction with such measurements.

Another important observation is that while gas emissions, which may be used for an indication of hypoglycemia and/or an indication of hyperglycemia, are in large parts provided through exhalation, there is enough gas emissions also from the skin so that a reliable detector can be provided. Moreover, it has been observed that while it may take a longer time for the gas emissions to be transmitted from the skin than from exhalation, they can still be detected quickly and reliably enough for the detector to be able to provide an actionable alarm for hypoglycemia and/or hyperglycemia.

In an embodiment, the detector comprises one or more flanges (herein also “the flanges”) for supporting an adhesive patch for attaching the detector to the skin. This allows the detector to be easily used as it can be attached to skin for an extended period of time during which it may repeatedly perform measurements for detection of hypoglycemia and/or hyperglycemia. During use of the detector, the flanges can be positioned to form a substantially uniform plane with the skin for attaching an adhesive patch so that the grip of the adhesive patch is maintained both with respect to the skin and to the detector.

In an embodiment, the one or more flanges comprise elastic material for bending the one or more flanges against the skin. This allows the attachment of the flanges to the skin to be improved. Also, this may be used to make wearing the detector more comfortable.

In an embodiment, the enclosure comprises an elevated portion, such as a cover, for elevated positioning with respect to the one or more flanges and the one or more flanges are positioned at least partially surrounding the elevated portion. This allows one or more of the electronic components of the detector, such as the sensors and/or the processors, to be fitted within the elevated portion for elevated positioning with respect to the flanges, while the flanges can still a surface for attachment to the skin. This allows a compact detector to be formed. The detector may still be attached to the skin utilizing the adhesive patch, which may define an opening for accommodating the elevated portion. This substantially reduces the bending of the adhesive patch when used to attach the detector to the skin.

In an embodiment, the bottom surface is made of biocompatible material, such as biocompatible plastic. This allows the detector to be maintained against the skin for an extended period of time. The detector may be worn for several hours at a time, for example from morning to evening, or even one or more days at a time. When the parts of the detector maintaining contact with the skin during use are of biocompatible material, any irritation caused by the detector may be mitigated or removed altogether.

In an embodiment, the one or more gas sensors comprise a metal-oxide semiconductor (MOS) sensor. This has been found to allow reliable and cost-effective detection of gases relevant for hypoglycemia and/or hyperglycemia. Such gases include VOC gases and the MOS sensor can therefore also be a VOC sensor.

In an embodiment, the one or more gas sensors are configured for detecting at least acetone and/or isoprene emissions. Sensors sensitive to these gases have been found to allow improved detection for hypoglycemia and/or hyperglycemia.

In an embodiment, the one or more measurement signals are indicative of resistance for the one or more gas sensors. It has been found that this allows an efficient measure for the measurement signal, from which measure hypoglycemia and/or hyperglycemia can still be detected.

In an embodiment, the one or more processors are configured to detect the indication of hypoglycemia and/or the indication of hyperglycemia by obtaining one or more measurement values from the one or more measurement signals and comparing the one or more measurement values to one or more baseline values indicative of a non-hypoglycemia and/or non-hyperglycemia state, i.e. the absence of hypoglycemia and/or hyperglycemia. This has been found to allow an efficient detection of hypoglycemia and/or hyperglycemia without actually measuring the blood sugar level. The one or more baseline values may be determined from a measurement of a user, such as a diabetic person, when the user is not in hypoglycemia and/or hyperglycemia. Then, at any time one or more measurements may be performed with the detector for the same user, providing one or more measurement values. A comparison of these values may then be performed to provide an indication of hypoglycemia and/or an indication of hyperglycemia. For example, if a measurement value differs from the baseline value by a threshold value such as a threshold percentage, an indication of hypoglycemia and/or an indication of hyperglycemia may be provided. Since the detector allows hypoglycemia and/or hyperglycemia to be detected as a clear change with respect to non-hypoglycemia and/or non-hyperglycemia state, the threshold value can be used to correspond the blood sugar level indicative of hypoglycemia, such as 3.9-4 mmol/l, and/or the blood sugar level indicative hyperglycemia, such as 11-11.1 mmol/1 or 7.0 mmol/l.

In an embodiment, the one or more measurement signals correspond to one or more measurements over a period of time. This allows a temporal measurement profile to be used and it has been found to allow notable improvement in accuracy for detecting hypoglycemia and/or hyperglycemia.

In an embodiment, the detector is configured to operate the one or more gas sensors utilizing temperature cycling for detecting gas emissions from the skin. This has been found to allow notable improvement in the sensitivity of the sensors for hypoglycemia and/or hyperglycemia detection, while allowing the sensors to be compactly packed within the enclosure. Moreover, temperature cycling allows maximizing the sensitivity of the sensor for particular gases or gas mixtures for detecting hypoglycemia and/or hyperglycemia. For temperature cycling, the sensors may be cyclically heated and cooled. The heating and/or cooling may be performed rapidly, even substantially instantaneously, for improved sensitivity. In some embodiments, it has been found that heating and/or cooling period of up to 30-60 seconds may be used for a particularly improved sensitivity for hypoglycemia and/or hyperglycemia detection. Cooling, in particular, can be very rapid, for example with a cooling period of less than one second, to allow improved sensitivity. Similarly, a heating period may range, for example, from 1 millisecond to 10 seconds. It has also been found that a heating up to 400-500 Celsius can be utilized efficiently for particularly improved sensitivity for hypoglycemia and/or hyperglycemia detection for a detector as described herein.

In an embodiment, the detector is configured to provide the one or more measurement signals during one or more cooling phases of the temperature cycling. This has been found to particularly improve sensitivity of the sensors for hypoglycemia and/or hyperglycemia detection. A cooling phase includes at least the cooling period, where the temperature of the sensors is decreased but it may additionally include an additional recovery period for the sensors, during which the response of the sensors returns towards a steady-state response. While the initial cooling period may be less than a second, the subsequent additional recovery period may last for more than a second, for example 5-10 seconds or more, allowing the measurement signals to be provided over a period of time, for example 2-5 seconds or more.

In an embodiment, the detector is configured for providing the alarm signal to a remote device and/or as a local alarm signal for sensory perception. The detector may comprise a wireless transmitter, for example as a part of a transmitter-receiver, for transmitting the alarm signal to a remote device, such as a mobile phone. Alternatively or additionally, the detector may comprise an alarm such as a an audible and/or vibration alarm for providing a local alarm for sensory perception. This in particular removes the need for any application interfaces to be operated, for example a mobile-phone application to be installed, for being able to utilize the detector, improving the ease of use.

According to a second aspect, an arrangement comprises the hypoglycemia and/or hyperglycemia detector according to the first aspect or any of its embodiments alone or in any combination. The arrangement also comprises an adhesive patch for attaching the detector to the skin. The adhesive patch may be separable from the detector, allowing the adhesive patch to be replaced while the use of the detector is continued.

In an embodiment, the adhesive patch defines an opening for accommodating an elevated portion of enclosure of the detector. This substantially reduces the bending of the adhesive patch when used to attach the detector to the skin. The opening allows the adhesive patch to at least partially surround the elevated portion, thereby improving the attachment of the adhesive patch to the detector and to the skin. Simultaneously, it allows the adhesive patch to be positioned to form a substantially uniform plane with the skin for attaching the detector so that the grip of the adhesive patch is maintained both with respect to the skin and to the detector.

It is to be understood that the aspects and embodiments described above may be used in any combination with each other. Several of the aspects and embodiments may be combined together to form a further embodiment of the invention.

### BRIEF DESCRIPTION OF THE DRAWINGS

The accompanying drawings, which are included to provide a further understanding and constitute a part of this specification, illustrate examples and together with the description help to explain the principles of the disclosure. In the drawings:

FIG. 1a illustrates a detector according to an example in a cross-sectional view from one side,

FIG. 1b illustrates a detector according to an example in an exploded view,

FIG. 2 schematically illustrates detection of hypoglycemia and/or hyperglycemia according to an example, and

FIG. 3 schematically illustrates measurement values for detection of hypoglycemia and/or hyperglycemia according to an example.

Like references are used to designate equivalent or at least functionally equivalent parts in the accompanying drawings.

### DETAILED DESCRIPTION

The detailed description provided below in connection with the appended drawings is intended as a description of examples and is not intended to represent the only forms in which the example may be constructed or utilized. However, the same or equivalent functions and structures may be accomplished by different examples.

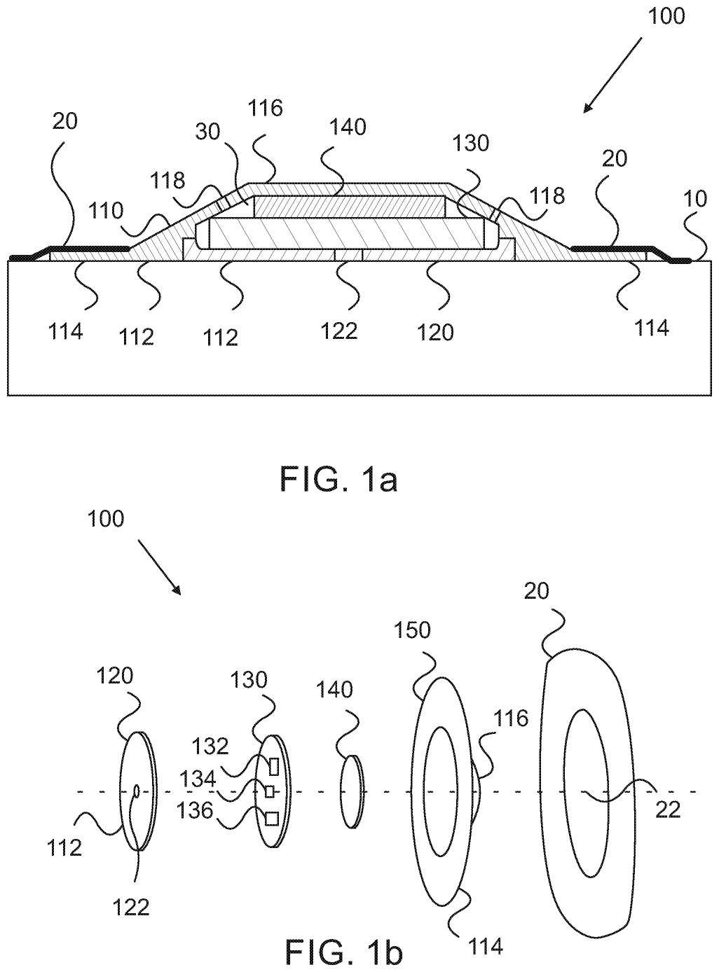

FIGS. 1a and 1b show an example of a hypoglycemia and/or hyperglycemia detector 100 (herein also “the detector”) in a cross-sectional side view and in an exploded view, respectively.

In use, the detector 100 can be positioned on skin 10 for hypoglycemia and/or hyperglycemia detection. In some embodiments, the detector 100 is configured for positioning on the arm of a person for hypoglycemia and/or hyperglycemia detection as this has been found to allow comfortable and precise monitoring. Other positionings are also possible, for example on the leg or the torso of a person. The detector 100 can be configured for being maintained on the skin 10 for hypoglycemia and/or hyperglycemia detection for an extended period of time involving a plurality of measurement cycles. For this purpose, the detector 100 may be configured for being attached to the skin 10 by an adhesive patch 20 such as a plaster. This allows accurate positioning of the detector 100 easily together with maintaining the positioning during the use of the detector 100. This, in turn, allows increased measurement precision since repeated measurements, which may include an initial calibration measurement, can be performed at the same position with respect to the skin 10. However, the detector 100 may also be repositioned during use since it can easily be reattached. This way, any irritation caused by the detector 100 on the skin may be reduced. If necessary, the detector 100 may be recalibrated when repositioning. Alternative or additionally to attachment with an adhesive patch 20, the detector 100 may comprise or be configured to be attached to a strap, such as an arm band or a wrist band, for strapping attachment of the detector 100 with respect to the skin 10, for example to an arm or a wrist of a person using the detector 100.

The detector 100 comprises an enclosure 110, which can be configured for positioning against the skin 10 for hypoglycemia and/or hyperglycemia detection. The enclosure 110 comprises a bottom surface 112 configured for positioning against the skin 10 for hypoglycemia and/or hyperglycemia detection. The bottom surface 112 may be configured to allow gas emissions from the skin 10 into the enclosure 110. The bottom surface 112 may comprise one or more holes 122, such as through-holes, for directing the gas emissions from the skin 10 into the enclosure 110. Alternatively or additionally, the bottom surface 112 may comprise a gas-permeable surface such as a gas-permeable membrane for allowing the gas emissions from the skin 10 to enter the enclosure 110. The bottom surface 112 may be flat. The enclosure 110 may comprise one or more holes 118 for allowing air circulation through the enclosure 110. The enclosure 110 may comprise or be made of plastic, for example biocompatible plastic. It is noted that any or all parts of the detector 100, such as the bottom surface 112, maintaining contact with the skin 10 while the detector 100 is in use may be made of biocompatible material such as biocompatible plastic, thereby mitigating any irritation caused by prolonged use of the detector 100. In any case, the enclosure 110 and/or the detector 100 as a whole may be waterproof.

The enclosure 110 may be composed of one or more parts. As an example, the enclosure 110 may comprise a bottom cover 120, such as a bottom plate, which may be detachable for providing a hatch for the enclosure 110. The bottom cover 120 may at least partially form the bottom surface 112. The bottom cover 120 may comprise one or more of the one or more holes 122, for example at the center of the bottom cover 120. The enclosure 110 may also comprise an elevated portion 116, such as a cover, for elevated positioning with respect to the bottom surface 112. The elevated portion 116 may be arranged for separable coupling with the bottom cover 120 or they may together constitute a single monolithic part. As an example, the bottom cover 120 may be circular, in which case it may be configured also for screw-in coupling, directly or indirectly, to the elevated portion 116. Similarly, the elevated portion 116 may have a circular shape but other shapes are possible as well, for example an oval shape or a rectangular shape. With circular shape, the elevated portion may be configured for screw-in coupling, directly or indirectly, to the bottom cover 120. In addition, it may allow an adhesive patch 20 defining a circular opening 22 to be used for easy and reliable attachment of the detector 100 to the skin 10.

The detector 100 may comprise one or more flanges 114 (herein also “the flanges”), which can be configured for supporting an adhesive patch 20 for attacking the detector to the skin 10. In some embodiments, the flanges 114 may be formed as a monolithic part with respect to the enclosure 110, wherein the enclosure itself may be formed as a single monolithic part or as multiple parts. For example, the flanges 114 may be formed as a monolithic part with the elevated portion 116 of the enclosure 110, such as the cover, and/or with the bottom cover 120 of the enclosure 110. In one convenient structure, the flanges 114 and the elevated portion 116 are coupled together, for example as a single monolithic part, for forming a top cover 150 of the detector 100. The flanges 114 may comprise or be formed of a material that is biocompatible material, such as biocompatible plastic, and/or that is same as a material of the enclosure 110, for example that of the elevated portion 116 and/or the bottom cover 120. Alternatively or additionally, the flanges 114 may comprise or be formed of an elastic material for bending the flanges 114 against the skin 10. The flanges 114 can be thin so that the adhesive patch can be applied across the flanges 114 and the skin 10 in a substantially uniform plane for attachment. With “substantially uniform” it should be understood that while slight changes in the elevation of the adhesive patch may still be visible, particularly where the adhesive patch 20 crosses from the flanges 114 to the skin 10, the flanges 114 are still thin enough for the plane to remain substantially uniform for the attachment. While the flanges 114 may have a substantially flat bottom and/or top for attachment, some texture may also be provided for attachment. The flanges 114 may be positioned around the enclosure 110 for providing an attachment surface for the adhesive patch 20. As an example, the flanges 114 may, continuously or discontinuously, encircle the enclosure 110, or its elevated portion 116, thereby surrounding it. The elevated portion 116 such as a cover may be configured for elevated positioning with respect to the flanges 114, in particular. While the illustration in FIG. 1b involves a single, continuous flange 114 continuously encircling the elevated portion 116, the detector 100 may also comprise two separate flanges 114, for example on the opposite sides of the enclosure 110 or the elevated portion 116 thereof.

The detector 100 may be made small and/or light for convenient use. It may be configured for fitting within a diameter of 10 centimeters when in use, for example within a diameter of 3-5 centimeters, wherein the diameter may be measurable in the plane of the skin 10. The detector 100 may be configured for fitting within a height of 2-3 centimeters when in use, even within a height of 5-10 millimeters, wherein the height may be measurable perpendicular to the plane of the skin 10. For a light embodiment, the detector 100 may have a weight of 100 grams or less, even 5-10 grams or less.

The enclosure 110 defines an interior space 30, where one or more components, such as electrical components, of the detector 100 may be positioned. The detector 100 comprises one or more gas sensors 132 (herein also “the sensors”), which are positioned within the enclosure 110. The enclosure 110 may be configured to, at least partially, prevent ambient gases from reaching the sensors 132. However, as noted above, the enclosure 110 may also be configured for air circulation through the enclosure 110. Importantly, the sensors 132 are configured for detecting gas emissions from the skin 10 for providing one or more measurements signals (herein also “the measurement signals”) for hypoglycemia and/or hyperglycemia detection. Correspondingly, the sensors 132 are positioned and configured so that the ambient gases do not prevent the detection of hypoglycemia and/or hyperglycemia. For this purpose, the sensors may be also directed towards the gas emissions from the skin, for example directly towards the bottom surface 112. Alternatively or additionally, the enclosure 110 may be configured for directing the gas emissions from the skin 10 towards the sensors 132, for example directly through the bottom surface 112 or through one or more channels of entry into the enclosure 110.

The sensors 132 may comprise one or more metal oxides, which change their electrical properties when exposed to gas emissions. The one or more metal oxides may comprise tin oxide (SnO₂) allowing wide reactivity and strong changes in resistance for hypoglycemia and/or hyperglycemia detection. The sensors 132 may comprise one or more digital sensors. The sensors 132, and the detector 100, may be configured for hypoglycemia and/or hyperglycemia detection in an ambient temperature, for example corresponding to 0-40 degrees Celsius, or room temperature, in particular. The sensors 132 may comprise one or more metal-oxide semiconductor (MOS) sensors, for example Bosch BME680 sensors. The sensors 132 may be VOC sensors. As an example, the sensors may be sensitive to at least acetone and/or isoprene emissions for providing the measurement signals for hypoglycemia and/or hyperglycemia detection. In some embodiments, the measurement signals are indicative of resistance for the sensors 132. For example, the measurement signals 132 may directly correspond to the resistance and/or the conductance for the sensors 132. In general, the measurement signals provided from the sensors 132 are different when the gas emissions from the skin 10 correspond to hypoglycemia and/or hyperglycemia and when the gas emissions from the skin 10 correspond to a non-hypoglycemia and/or non-hyperglycemia state. It is not necessary to know the exact difference, only that there is a difference. Since the difference can be straightforwardly verified for different sensor configurations, for example through clinical trials, the hypoglycemia and/or hyperglycemia detector 100 and any processors 134 therein can be configured depending on the actual sensor configuration utilized.

The detector 100 also comprises one or more processors 134 (herein also “the processors”). In particular, the processors 134 may comprise one or more microcontroller units (MCU), allowing a very cost-efficient solution for a cheap, easy-to-use hypoglycemia and/or hyperglycemia detector. Alternatively or additionally, the processors 134 may comprise one or more microprocessors. The detector 100 may also comprise a system-on-chip (SoC) comprising one or more of the processors 134.

In general, the processors 134 may comprise, for example, one or more of various processing devices, such as a coprocessor, a microprocessor, a controller, a digital signal processor (DSP), a processing circuitry with or without an accompanying DSP, or various other processing devices including integrated circuits such as, for example, an application specific integrated circuit (ASIC), a field programmable gate array (FPGA), a microcontroller unit (MCU), a hardware accelerator, a special-purpose computer chip, or the like.

The detector 100 may further comprise one or more memories (herein also “the memories”). The processors 134 may be configured to perform any of the processes described herein for the processors 134 according to program code comprised in the memories. The memories may be configured to store, for example, computer programs and the like. The memories may include one or more volatile memory devices, one or more non-volatile memory devices, and/or a combination of one or more volatile memory devices and non-volatile memory devices. For example, the memories may be embodied as magnetic storage devices (such as hard disk drives, floppy disks, magnetic tapes, etc.), optical magnetic storage devices, and semi-conductor memories (such as mask ROM, PROM (programmable ROM), EPROM (erasable PROM), flash ROM, RAM (random access memory), etc.).

The processors 134 and/or the memories may be arranged within the enclosure 110, for example within the elevated portion 116 of the enclosure 110.

Functionality described herein may be implemented via the various components of the detector 100. For example, the memories may comprise program code for performing any functionality disclosed herein or causing any functionality disclosed herein to be performed, and the processors 134 may be configured to perform the functionality, or cause the functionality to be performed, according to the program code comprised in the memory. When the detector 100 is configured to implement some functionality, some component and/or components of the detector 100, such as the processors 134 and/or the memories, may be configured to implement this functionality. Furthermore, when the processors 134 are configured for implementing some functionality, this functionality may be implemented using program code comprised, for example, in the memories. For example, if the detector 100 is configured for performing an operation, such as detecting an indication of hypoglycemia and/or an indication of hyperglycemia from the measurement signals for providing an alarm signal when the indication of hypoglycemia and/or the indication of hyperglycemia is detected, the memories and the computer program code can be configured to, with the processors 134, cause the detector 100 to perform that operation.

In addition, the detector 100 may comprise one or more electronic components 136. For example, the detector 100 may comprise a transceiver. The transceiver may be configured to, for example, transmit and/or receive data using, for example, a 3G, 4G, 5G, LTE, Bluetooth or WiFi connection. The processors 134 may be configured for causing the transceiver to transmit an alarm signal to a remote device such as a mobile phone, a wearable electronic device or any other kind of computing device. Alternative or additionally, the detector 100 may comprise an alarm for providing a local alarm signal for sensory perception. The local alarm signal may comprise an auditory signal and/or a haptic signal, for example a vibration signal. Alternatively or additionally, the local alarm signal may comprise a visual signal. Correspondingly, the alarm may comprise, alone or in any combination, a speaker for the auditory signal, a light for the visual signal and/or a haptic actuator for the haptic signal. The processors 134 may be configured for causing the alarm to produce the local alarm signal. The detector 100 may also comprise a power source 140 for providing electricity to the processors 134 and/or the sensors 132. The power source 140 may also be configured for providing electricity to any of the electronic components 136. The power source 140 may be replaceable or fixed. For replacing the power source 140, the detector 100 may comprise a detachable cover such as the bottom cover 120 and/or the top cover 150. In particular, it has been found that the detachable cover may be efficiently provided as part of the elevated portion 116, when the elevated portion is configured as a cover for the enclosure 110.

The detector 100 may comprise a support 130 for electronic components, which may comprise a circuit board such as a printed circuit board (PCB). The sensors 132 may be arranged on the support 130. Similarly, the processors 134 and/or the memories may be arranged on the support 130.

The support 130 and/or the power source 140 may be arranged within the enclosure 110, for example within the elevated portion 116 of the enclosure 110. As an example, the detector 100 may comprise a top cover 150 and a bottom cover 120, which may be part of the enclosure 110. The detector 100 may further comprise the support 130, for example an integrated circuit board, and/or the power source 140, for example a battery 140. The support 130 and/or the battery 140 may be arranged for positioning between the bottom cover 120 and the top cover 150, for example within an elevated portion 116 of the enclosure 110. The top cover 150 and/or the bottom cover 120 may be detachable, for example for replacement of the power source 140.

An arrangement comprises the detector 100 and the adhesive patch 20, such as a plaster. The detector 100 and the adhesive patch are configured for the detector to be attached to the skin 10 by the adhesive patch 20. For this purpose, the detector 100 and the adhesive patch 20 may have a complementary shape. In particular, the detector 100 may comprise the flanges 114 for supporting the adhesive patch 20 for attaching the detector 100 to the skin. Moreover, the enclosure 110 of the detector 110 may comprise the elevated portion 116 and the adhesive patch 20 may define an opening 22, such as a through-hole, for accommodating the elevated portion 116. The adhesive patch 20 may thereby be made to contact the detector 114 predominantly, or even solely, to the flanges 114 for attachment between the adhesive patch 20 and the detector 100. The opening 22 may be positioned at any place within the adhesive patch 20, for example at the center of the adhesive patch 20.

As an example, the adhesive patch 20 comprises an opening 22, such as a through-hole, or a cutout for the opening 22. The opening 22 may have a circular shape but other shapes are possible as well, for example an oval shape or a rectangular shape. The adhesive patch 20 may comprise or be made of biocompatible material for attachment to the skin 10. The adhesive patch 20 may be disposable so that it may be replaced while the use of the detector 100 is continued. Use of the adhesive patch 20 for attachment allows the position of the detector 100 to be changed during use so that irritation to the skin 10 may be reduced. For example, the adhesive patch 20 may be changed daily. The adhesive patch may be breathable and/or waterproof. It may be siloxane-free for minimizing contamination of the sensors 132 from siloxane for hypoglycemia and/or hyperglycemia detection. Similarly, it may be VOC-free for minimizing contamination of the sensors 132 from VOC. The adhesive patch 20 may be glue-free. The adhesive patch 20 may be customizable, for example having a customizable pattern.

The processors 134 are configured for detecting an indication of hypoglycemia and/or an indication of hyperglycemia from the measurement signals, directly or indirectly, for providing an alarm signal when the indication of hypoglycemia and/or the indication of hyperglycemia is detected. The processors 134 may be configured for causing the alarm signal to be provided, for example by utilizing the transceiver and/or the alarm. As an example, the indication(s) may be detected by obtaining one or more measurement values (herein also “the measurement values”) from the measurement signals and comparing the measurement values to one or more baseline values (herein also “the baseline values”) indicative of a non-hypoglycemia and/or non-hyperglycemia state. As the measurement signals can be indicative of resistance for the sensors 132, the measurement values may also correspond to resistance for the sensors 132. As an example, the measurement values may comprise one or more values corresponding to resistance for the sensors 132 at one or more points or periods in time. The measurement values may be processed from the measurement signals, for example as accumulated values and/or average values. In some embodiments, the measurement values correspond to an integral of resistance for the sensors 132 over one or more periods of time. A change, such as a drop or an increase, in such integral may then be obtained as an indication of hypoglycemia and/or an indication of hyperglycemia. In general, the indication of hypoglycemia and/or the indication of hyperglycemia may be obtained when the measurement values indicate a difference to the baseline values, for example a difference larger than a threshold difference. The threshold difference may correspond to a percentage of the baseline value, for example 10-20 percent thereof or more. It has been found that rather high values, such as 50-70 percent or more may also be used, as the change indicative of hypoglycemia and/or hyperglycemia may be large. The detector 100 does not need to measure the actual blood sugar level as it can reliably detect an indication of hypoglycemia and/or an indication of hyperglycemia from the gas emissions from the skin 10, which themselves are indicative of hypoglycemia and/or hyperglycemia. The indication of hypoglycemia and/or the indication of hyperglycemia may correspond, for example, a drop in resistance for the sensors 132.

In some embodiments, the detector 100 is configured, for example by utilizing the processors 134, to operate the sensors 132 utilizing temperature cycling for detecting gas emissions from the skin 10. For temperature cycling, the sensors 132 are rapidly heated up and cooled down for detection allowing sensitivity of the sensors 132 to be improved. An example of temperature cycling is provided by Schutze et al. (Environments 2017, 4, 20). While Schutze et al. relates to monitoring indoor air quality, it has been found that similar principles may be used also herein for detecting hypoglycemia and/or hyperglycemia. For this purpose, the sensors 132 may comprise, for example, a MOS sensor, which may also be a VOC sensor. In particular, the detector 100 may be configured, for example by utilizing the processors 134, to provide the measurement signals during one or more cooling phases of the temperature cycling. Utilizing the sensors 132 during the one or more cooling phases for providing the measurement signals has been found to allow improved detection of hypoglycemia and/or hyperglycemia.

The detector 100 may be configured, for example by utilizing the processors 134, for performing a calibration measurement for determining the one or more baseline values indicative of a non-hypoglycemia and/or non-hyperglycemia state. This allows user-specific calibration for the detector 100, which may be used to improve accuracy of the detector 100. A deviation from the baseline values may be used as an indication of hypoglycemia and/or an indication of hyperglycemia. Such a deviation may correspond to a change in resistance of the sensors 132, for example a decrease in resistance.

The detector 100 may be configured, for example by utilizing the processors 134, for providing the one or more measurement signals according to a schedule, for example at least once an hour. In an example, this is done at least once each 15-30 minutes. Each time, one or more measurements may be made, where several measurements may be used, for example, for improving accuracy by averaging. This allows also mitigating any inaccuracy that may result from the movement of the adhesive patch 20 with respect to the skin 10. A measurement or a measurement cycle for detecting a single indication of hypoglycemia and/or hyperglycemia may be performed in 30-60 seconds or faster, for example in 5-10 seconds or even faster.

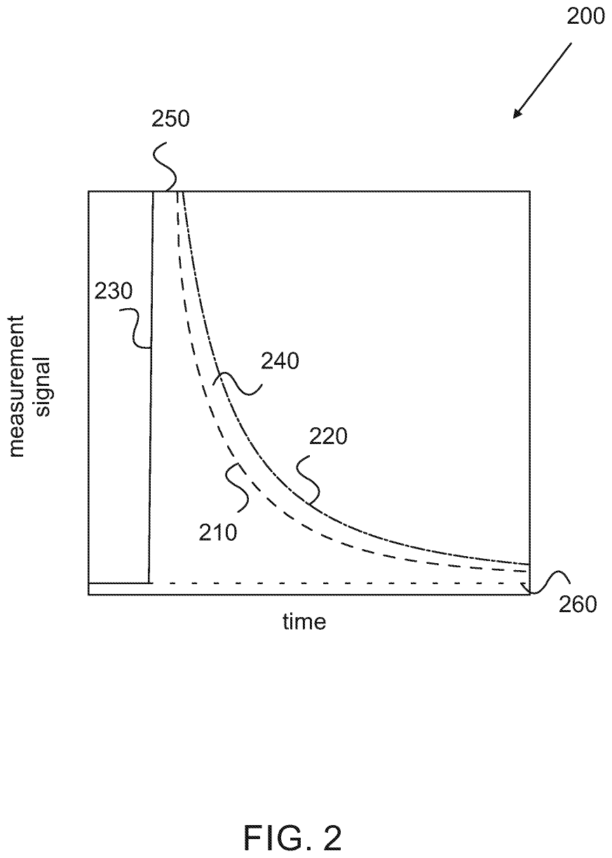

FIG. 2 shows an example for detection of hypoglycemia and/or hyperglycemia. While the example is specifically illustrated in terms of hypoglycemia, a similar example can be given for hyperglycemia as well. Therefore, any references to hypoglycemia herein may be considered to include, additionally or alternatively, also hyperglycemia. The example is illustrated in terms of a graph 200 corresponding to a measurement cycle, where a measurement signal, such as resistance of the sensors 132, is provided (vertical axis) as a function of time (horizontal axis). Measurement signal over a period of time is illustrated both when the user of the detector 100 is in hypoglycemia state 210 and when the user of the detector 100 is in non-hypoglycemia state 220. In the illustration, an effect of temperature cycling is visualized as dynamic changes for both states. However, the example may also be considered for a detector 100 not utilizing temperature cycling.

For both states, the measurement signal, for example corresponding to the resistance of the sensors 132, first has a rapid increase 230 when the temperature of the sensors 132 is increased. This corresponds to an increase in the sensitivity of the sensors 132. For both states, the measurement signal also has a rapid decrease 210, 220 when the temperature of the sensors is decreased. The steepness of the decrease may, however, correspond to the amount of gas emissions detected, for example so that a more rapid decrease indicates a larger amount of gas emissions detected. Correspondingly, in hypoglycemia state 210 the decrease for the measurement signal may be more rapid than in non-hypoglycemia state 220. The difference between the measurement signal for the hypoglycemia state 210 and the measurement signal for the non-hypoglycemia state 220 may be used for detecting hypoglycemia, for example if the difference 240 is larger than a threshold value. The threshold value may, for example, correspond to a percentage of the measurement signal in non-hypoglycemia state 220. It is noted that while the sensors 132 may reach saturation 250 at one or more points in time, this is not necessarily any problem since this could simply be interpreted as the measurement signals for the two states being equal. The measurement may in any case be performed so that at least one point in time or a time period is situated after the saturation 250. It is also noted that while the cooling period initiating the decrease for the measurement signals may be short, for example less than one second and coinciding with the saturation 250, the cooling phase during which the measurement signals for detecting the indication of hypoglycemia (and/or the indication of hyperglycemia) can be provided may be larger, for example comprising the whole illustrated period after the saturation 250. As an example, in FIG. 2 the illustrated horizontal axis may correspond to a time period of 30 seconds with the sensors 132 in a steady state 260 during a time period of 0-4 seconds from the beginning. A rapid heating period of 1 second initiated at 4 seconds from the beginning may then correspond to the increase 230, whereas a rapid cooling period of less than 1 second initiated directly thereafter takes place during the saturation 250. The cooling phase may here comprise the period of 5-30 seconds from the beginning as the response of the sensors 132 recovers towards the steady state 260. An indication for hypoglycemia and/or an indication of hyperglycemia may be obtained from the accumulated or averaged difference between the measurement signals for the two states 210, 220 over a time period such as 0-30 seconds, 5-25 seconds or 10-15 seconds, for example.

As an important example, the one or more measurement signals may correspond to one or more measurements over a period of time so that the indication of hypoglycemia and/or the indication of hyperglycemia may correspond a compound value for the one or more measurement signals over time, for example a cumulated and/or averaged difference for the measurement signals in hypoglycemia and/or hyperglycemia state 210 and the measurement signals in non-hypoglycemia and/or non-hyperglycemia state 220 over a period of time. Naturally, this principle may be utilized to generate indicators with various different kinds of details. In any case, any single measurement cycle, for example as illustrated in FIG. 2, corresponding to one or more measurements over a period of time, may be represented by a single scalar value, where the scalar value corresponds to the measurement signals over a period of time.

For a detector 100 not utilizing temperature cycling, the same principles may be utilized with the distinction that in such case the measurement signal in non-hypoglycemia and/or non-hyperglycemia state 220 may remain substantially constant over time. Correspondingly, the measurement signal in hypoglycemia and/or hyperglycemia state 210 may correspond to smaller values but may also be substantially constant or change in accordance with any changes in the gas emissions from the skin 10. Also here, the indication of hypoglycemia and/or the indication of hyperglycemia may correspond a compound value for the one or more measurement signals over time, for example a cumulated and/or averaged difference as indicated above.

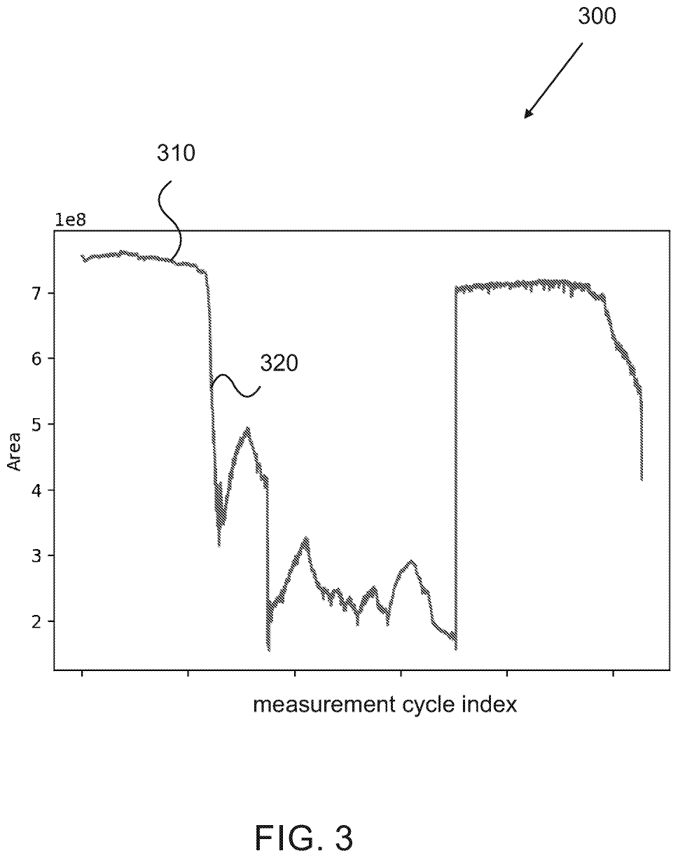

FIG. 3 shows an example of measurement values for detection of hypoglycemia and/or hyperglycemia. Also here, the example is specifically illustrated in terms of hypoglycemia but a similar example can be given for hyperglycemia as well. Therefore, any references to hypoglycemia herein may be considered to include, additionally or alternatively, also hyperglycemia. Here, the measurement values correspond to the area under the curve for the measurement signals as a function of time, for example as illustrated in FIG. 2. The graph 300 thereby illustrates such measurement values as a function of an index for the measurement cycle. Importantly, each point on a curve 310 of the graph 300 thereby corresponds to a single measurement cycle, where the point may be obtained, for example, as a scalar value corresponding a cumulated difference between the measurement signal in the two states as illustrated in FIG. 2 over some time period during the cooling phase of the sensors 132. A change 320, such as a drop, in the curve 310 may be used as an indication that the user of the detector 100 is in hypoglycemia and/or hyperglycemia state.

Measurements in accordance with the illustrations of FIGS. 2 and/or 3 may also be used for screening sensor configurations for the detector 100. Sensor configurations providing a clear indication for hypoglycemia and/or hyperglycemia, such as a clear difference 240 or a clear change 320, may be utilized and suitable sensor configurations may be selected based on the application requirements, for example cost-efficiency requirements.

The different functions discussed herein may be performed in a different order and/or concurrently with each other.

Any range or device value given herein may be extended or altered without losing the effect sought, unless indicated otherwise. Also any example may be combined with another example unless explicitly disallowed.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item may refer to one or more of those items.

The term ‘comprising’ is used herein to mean including the method, blocks or elements identified, but that such blocks or elements do not comprise an exclusive list and a method or apparatus may contain additional blocks or elements.

Expressions such as ‘plurality’ are in this text to indicate that the entities referred thereby are in plural, i.e. the number of the entities is two or more.

Although the invention has been the described in conjunction with a certain type of apparatus and/or method, it should be understood that the invention is not limited to any certain type of apparatus and/or method. While the present inventions have been described in connection with a number of examples, embodiments and implementations, the present inventions are not so limited, but rather cover various modifications, and equivalent arrangements, which fall within the purview of the claims. Although various examples have been described above with a certain degree of particularity, or with reference to one or more individual embodiments, those skilled in the art could make numerous alterations to the disclosed examples without departing from the scope of this specification.

1. Hypoglycemia and/or hyperglycemia detector comprising:
an enclosure comprising a bottom surface configured for positioning against skin;
one or more gas sensors positioned with-in the enclosure and configured for detecting gas emissions from the skin for providing one or more measurement signals; and
one or more processors configured for detecting an indication of hypoglycemia and/or an indication of hyperglycemia from the one or more measurement signals for providing an alarm signal when the indication of hypoglycemia and/or the indication of hyperglycemia is detected;
characterized in that the detector is configured to operate the one or more gas sensors utilizing temperature cycling for detecting gas emissions from the skin, wherein the one or more gas sensors are cyclically heated and cooled.

2. The detector according to claim 1, comprising one or more flanges for supporting an adhesive patch for attaching the detector to the skin.

3. The detector according to claim 2, where-in the one or more flanges comprise elastic material for bending the one or more flanges against the skin.

4. The detector according to claim 2, wherein the enclosure comprises an elevated portion for elevated positioning with respect to the one or more flanges and the one or more flanges are positioned at least partially surrounding the elevated portion.

5. The detector according to claim 1, wherein the bottom surface is made of biocompatible material.

6. The detector according to claim 1, wherein the one or more gas sensors comprises a metal-oxide semiconductor (MOS) sensor.

7. The detector according to claim 1, wherein the one or more gas sensors are configured for detecting at least acetone and/or isoprene emissions.

8. The detector according to claim 1, wherein the one or more measurement signals are indicative of resistance for the one or more gas sensors.

9. The detector according to claim 1, wherein the one or more processors are configured to detect the indication of hypoglycemia and/or the indication of hyperglycemia by obtaining one or more measurement values from the one or more measurement signals and comparing the one or more measurement values to one or more baseline values indicative of a non-hypoglycemia and/or non-hyperglycemia state.

10. The detector according to claim 1, wherein the one or more measurement signals correspond to one or more measurements over a period of time.

11. The detector according to claim 1, wherein the detector (100) is configured to provide the one or more measurement signals during one or more cooling phases of the temperature cycling.

12. The detector according to claim 1, wherein the detector is configured for providing the alarm signal to a remote de-vice and/or as a local alarm signal for sensory perception.

13. An arrangement comprising the hypoglycemia and/or hyperglycemia detector according to any preceding claim and an adhesive patch for attaching the detector to the skin.

14. The arrangement according to claim 13, wherein the adhesive patch defines an opening for accommodating an elevated portion of the enclosure the detector.
